(19) United States
(12) Patent Application Publication
(10) Pub. No.: US 2007/0225203 A1
(43) Pub. Date: Sep. 27, 2007

(54) Method and Composition for Preventing Multiple Organ Dysfunction Syndrome

(75) Inventors: Klaske Van Norren, Renkum (NL); Eduard Christiaan Van Hoorn, Lelystad (NL); Robert Johan Joseph Hageman, Wageningen (NL); Houkje Bouritius, Zeist (NL); Adrianus Johannes Maria Vriesema, Houten (NL); Cornelus Johannes Petrus Van Limpt, Amersfoort (NL); Mirian Lansink, Houten (NL); Marieke Elise Van Meeteren, Hilversum (NL)

Correspondence Address: THE WEBB LAW FIRM, P.C., 700 KOPPERS BUILDING, 436 SEVENTH AVENUE, PITTSBURGH, PA, 15219, US

(73) Assignee: N.V. NUTRICIA, Zoetermeer (NL)

(21) Appl. No.: 10/572,239

(22) PCT Filed: Sep. 20, 2004
(86) PCT No.: PCT/NL04/00650

§ 371 Date: Mar. 1, 2007

(30) Foreign Application Priority Data
Sep. 19, 2003 (EP) 03077971.4

(51) Int. Cl. A61K 38/55; A61K 31/70; A61K 31/715; A61K 31/718; A61K 31/724; A61K 31/7076; A61K 31/525

(52) U.S. Cl. 514/2; 514/23; 514/46; 514/251; 514/54; 514/58; 514/59; 514/60

(57) ABSTRACT

One aspect of the present invention relates to a method of preventing multiple organ dysfunction syndrome in a mammal suffering from trauma, said method comprising enterally administering to said mammal, within 24 hours of the occurrence of the trauma, (i) digestible water soluble carbohydrates and (ii) a liver guanosine-5′-triphosphate (GTP) increasing component and/or peptides with Angiotensin Converting Enzyme (ACE) inhibiting activity. Another aspect of the invention relates to an aqueous liquid composition containing: −20-200 g/l digestible dissolved carbohydrates; −5-5000 mg/l guanosine equivalents in combination with 1-100 g/l ribose equivalents and/or 2-2000 mg/l flavonoides; or 0.01 to 10 mM of peptides with ACE inhibiting activity; and −45 to 97.95 wt. % water.

### TECHNICAL FIELD OF THE INVENTION

One aspect of the present invention relates to a method of preventing multiple organ dysfunction syndrome following trauma. The present method comprises enteral administration of a liquid nutritional composition shortly before and/or after the occurrence of a trauma.

Another aspect of the invention concerns a liquid nutritional composition for use in the aforementioned method.

### BACKGROUND OF THE INVENTION

With the advent of sophisticated monitoring systems and more effective single-organ support, the chances of patients being resuscitated from acute trauma are continuously increasing. However, following the “survival” of the initial phase of critical illness, these patients frequently progress into the clinical syndrome of Multiple Organ Dysfunction. MOD is characterized by a progressive deterioration and subsequent failure of the bodies physiological system¹. Because no effective treatments have been developed so far, MOD is associated with high mortality rates.

Multiple organ dysfunction is no longer viewed as a series of isolated failures. On autopsy, the involved organs display similar patterns of tissue damage although they are often remote from the initial injury site or septic source. This complex syndrome, once thought to be solely related to cardiovascular dysfunction and/or isolated organ failure, is now recognized as a systemic disturbance mediated by a sustained inflammatory response to injury, irregardless of the initiating factor(s). The multiple organ dysfunction syndrome attests to the complex interaction between organ systems in both their functioning and pathological states.

Several mechanisms have been postulated to be involved in post-ischemia induction of MOD. The gut-liver-lung axis has been associated to play a dominant role in the incidence and severity of this single and multiple organ dysfunction (S)MOD²⁻⁷. More specifically, the intestine is often referred to as the driving force of multiple organ dysfunction⁸⁻¹¹. The post-ischemic increase in reactive oxygen species can directly or indirectly (by macrophages and lymphocytes) activate neutrophils that subsequently can infiltrate at the site of inflammation causing tissue injury. These neutrophils have recently also been reported to increase paracellular transport in ileum. This damage of the intestinal barrier has often been mentioned to result in increased trans-epithelial bacterial transport and their endotoxins resulting in an inflammatory challenge of the patient, which has been reported to be involved in the incidence of MOD.

Recently, oxidative stress and neutrophil activation have been suggested as the two keystones of ischemia reperfusion injury¹². It is generally accepted that upon reperfusion (post-ischemia) a burst of ROS are released by several mechanisms, which may exceed the body's anti-oxidant capacity causing oxidative stress¹³⁻¹⁸ . Importantly, these ROS activate the inflammatory transcription factor NF-kB. Although an inflammatory response may be necessary, control of the inflammatory response is greatly lost after ischemia and therefore the pro-inflammatory cytokines TNFα and II-6 may be aggravated beyond their need. The importance of these ROS in NF-kB induction is for instance demonstrated by addition of N-acetylcystein, which upregulates glutathione levels in blood plasma, resulting in a decreased NFkB response and subsequently lowered TNFα¹².

Preoperative fasting has been reported to alter the morphological and metabolic responses to stress¹⁹⁻²¹ e.g. translocation of bacterial and their endotoxins has been reported to increase²²⁻²⁴. This increased translocation can be due to either decreased intestinal barrier function, a decreased hepatic function, especially the Kupfer cells P3 of the hepatic reticuloendothelial system (RES) or both. Moreover, dysfunction of the reticuloendothelial system (RES) system due to intestinal ischemia has been reported, especially in fasted animals²⁵⁻²⁸.

EP-A 0 564 511 discloses a beverage for preoperative intake consisting of an aqueous solution which is hypotonic (250-295 mOsm/kg) and contains 8-20 g of carbohydrates per 100 ml. The beverage may be used for suppressing the negative influence of a surgical operation on the post-operative carbohydrate metabolism of the patient and for improving the defence capacity of the patient upon bleeding in connection with or after surgery.

U.S. Pat. No. 5,438,043 describes a beverage for preoperative use, which comprises a hypotonic aqueous solution of between 8 and 20 grams of a carbohydrate mixture per 100 ml. The US patent describes a dry substance to be dissolved to yield 100 ml solution containing 11.7 g dextrin EP-A 0875 155 describes a liquid nutritional composition for peri-operational use which contains per 400 ml, 5-130 g soluble carbohydrates and 1-30 g glutamine or a glutamine precursor calculated as glutamine. The liquid composition is to be administered shortly before or after surgery to maintain anabolic metabolism without causing problems of anaesthesia and emptying of the stomach.

EP-A 0 302 807 describes liquid nutritionally balanced nourishing products which contain a source of amino nitrogen, carbohydrates, edible fats, minerals, vitamins and at least one nucleoside. Example IX discloses an aqueous liquid product is containing 7.32% maltodextrins and 0.15% nucleosides and/or nucleotides, said nucleosides and/or nucleotides containing 150 mg guanosine and/or 30 mg guanosine monophosphate.

### SUMMARY OF THE INVENTION

Before scheduled surgery, patients are usually subjected to fasting for at least 8 hours, up to 24 hours, for reasons of safety with regard to anaesthesia and for preventing regurgitation of the stomach content and aspiration. Also, following surgery or severe trauma, patients often will not consume any nutrients for 8 hours or more.

The inventors have unexpectedly discovered that there is a correlation between the incidence of MOD following trauma and reduced intake of digestible carbohydrates as a result of fasting during the period shortly before and/or after the occurrence of the trauma. Furthermore, the inventors have established that the risk of MOD may be reduced significantly by enterally administering shortly before or after the occurrence of the trauma, a substantial amount of digestible water soluble carbohydrates in the form of an aqueous liquid composition containing said digestible water soluble carbohydrates in combination with a liver guanosine-5′-triphosphate (GTP) increasing component and/or peptides with Angiotensin Converting Enzyme (ACE) inhibiting activity. Liver GTP increasing components that may advantageously be employed in accordance with the invention are guanosine equivalents and ribose equivalents.

The experimental data suggest that animals that are peri-operatively fed with a carbohydrate solution, as compared to fasted animals, develop significantly less intestinal permeability and also suffer from much less translocation of bacteria to liver, kidney and mesenteric lymphnodes. These data are further supported by biochemical characterizations of oxidative stress per organ and energy status of the liver.

Although the inventors do not wish to be bound by theory it is believed that the mechanism behind the protective effect of peri-operative administration of digestible carbohydrates on the incidence of MOD is somehow associated with the effect of said administration on both the intestine and the liver. The results indicate that the present method supports the maintenance of the gut barrier function after trauma.

Having established the relation between essential liver functioning and MOD, the inventors have additionally discovered that the prophylactic effect of the present liquid composition on MOD is further enhanced by incorporating into said composition an effective amount of one or more components capable of increasing liver guanosine-5′-triphosphate (GTP). The inventors have discovered an inverse relation between liver GTP and the incidence of MOD. Liver GTP levels may be increased effectively in accordance with the present invention by administering guanosine, ribose and/or precursors of these component(s).

Increased plasma levels of asymmetric dimethylarginine (ADMA) are also deemed to constitute an additional risk factor for MOD. It was found that the inclusion of an effective amount of peptides with ACE inhibiting activity in the present liquid composition will help to prevent that plasma concentrations of ADMA reach undesirably high levels.

### DETAILED DESCRIPTION OF THE INVENTION

Accordingly, one aspect of the invention is concerned with a method of preventing multiple organ dysfunction syndrome in a mammal suffering from trauma, said method comprising enterally administering to said mammal, within 24 hours of the occurrence of the trauma, (i) a liver GTP increasing component selected from the group consisting of: 2-2000 mg guanosine equivalents; 0.5-40 g ribose equivalents; and combinations thereof and (ii) at least 20 g of digestible water soluble carbohydrates in the form of an aqueous liquid composition containing at least 10 g/l, preferably at least 20 g/l of said digestible water soluble carbohydrates.

Another aspect of the invention relates to a method of preventing multiple organ dysfunction in a mammal suffering from trauma, comprising enterally administering to said mammal, within 24 hours of the occurrence of the trauma, (i) 0.05-100 mmole of peptides with ACE inhibiting activity, said peptides exhibiting an IC-50 concentration as defined in the specification of less than 1000 μM and (ii) at least 20 g of digestible water soluble carbohydrates in the form of an aqueous liquid composition containing at least 10 g/l of said digestible water soluble carbohydrates. The IC-50 concentration is a measure of the potency of a substance or composition to inhibit ACE activity and may be determined as described below under “Methods”.

The terminology “digestible carbohydrates” as used herein refers to carbohydrates that can either be absorbed as such by the gastrointestinal tract or that can be degraded by the gastrointestinal tract to absorbable components, provided said degradation does not involve fermentative degradation by the intestinal microflora.

The term “guanosine equivalents” as used in here, encompasses guanosine as well as salts of guanosine and precursors of guanosine, notably precursors that can liberate guanosine or a guanosine salt by in vivo conversion, e.g. hydrolysis, of the precursor molecule. Typical examples of guanosine precursors that can be hydrolysed to produce guanosine or a guanosine salt are guanosine esters.

The term “ribose equivalents” is defined in accordance with the definitions provided above for guanine equivalents and folic acid equivalents. Ribose equivalents may be administered in the form of synthetic or natural ribose or, for instance, as a precursor in the form of a nucleobase adduct, e.g. as a ribose guanosine adduct. Other suitable examples of ribose precursors include ribose esters.

The terminology “enterally administering” encompasses oral administration (including oral gavage administration) as well as rectal administration, oral administration being most preferred. Unless indicated otherwise, the dosages mentioned in this application refer to the amounts delivered during a single serving or single administration event. If the present composition is ingested from a glass or a container, the amount delivered during a single serving or single administration will typically be equal to the content of said glass or container.

Examples of trauma that can lead to MOD that can be treated prophylactically with the present method include surgery and major injuries such as burns, lesions and haemorrhage. The present method is particularly suitable for preventing MOD resulting from surgery, particularly prescheduled surgery. In case of, for instance, prescheduled surgery it is possible to administer the present liquid composition prior to the occurrence of the trauma Administration of the liquid composition prior to the occurrence of the trauma offers the important advantages that the composition can be administered simply by asking the patient to drink it and that the effect will be manifest when the actual trauma occurs.

The digestible carbohydrates employed in accordance with the invention may suitably include monosaccharides, disaccharides and polysaccharides. In a particularly preferred embodiment of the present invention the digestible water soluble carbohydrates are largely glucose based. In accordance with this embodiment said digestible water soluble carbohydrates optionally contain saccharides other than glucose in amounts of up to 6%, calculated on the molecular weight of the digestible carbohydrate. Examples of other saccharides that may occur in the digestible glucose based carbohydrates include D-fructose, D-arabinose, D-rhamnose, D-ribose and D-galactose, though preferably these saccharides are not located at the terminal side of the present carbohydrates. The glucose units of oligo- and polysaccharides are preferably predominantly connected via alpha 14 or alpha 1-6 bonds in order to be digestible. The digestible carbohydrates of the invention encompass both linear and branched oligo- and polysaccharides. The number of saccharide units is indicated via a number n. Oligosaccharides have a number of n between 3 and 10; polysaccharides between 11 and 1000 and preferably between 11 and 60.

Preferably, the present liquid composition contains between 30 and 200 g/l of digestible polysaccharides since, in comparison to monosaccharides and disaccharides, polysaccharides are absorbed more slowly. In another preferred embodiment, the composition contains a combination of polysaccharides and mono- and/or disaccharides. More preferably, the digestible carbohydrates comprise between 60-99 wt. % digestible oligo- and/or polysaccharide and between 140 wt. % digestible mono- and/or disaccharides. A suitable example of a digestible water soluble oligosaccharide is glucose syrup. Suitable examples of the digestible water soluble polysaccharides include dextrins, maltodextrins, starches, dextran and combinations thereof. Most preferably the water soluble polysaccharide contains at least 50 wt. %, more preferably at least 80 wt. % of polysaccharides selected from the group consisting of dextrin, maltodextrin and combinations thereof, dextrin being most preferred. In a particularly preferred embodiment the digestible carbohydrates include at least 1 wt. % monosaccharide, particularly at least 1 wt. % fructose. Typically, the digestible carbohydrates will contain not more than 20 wt. % fructose in monosaccharide form.

In a particularly preferred embodiment of the invention, the method comprises enterally administering, within 24 hours of the occurrence of the trauma, at least 50 g, more preferably at least 70 g of the digestible water soluble carbohydrates in the form of the aqueous liquid composition. The liquid composition may be administered as a single bolus or, alternatively, it may be administered in two or more doses during the 24 hour period. Preferably, the liquid composition is administered in at least 2 separate doses during the 24 hours period, the administration events preferably being at least 1 hour apart. A particularly suitable protocol comprises administering a sufficient amount of the present liquid composition during the period ranging from 24-8 hours prior to the trauma to deliver at least 40 g of digestible carbohydrates and to deliver at least 20 g of digestible carbohydrates during the period of 8-1 hour prior the trauma.

In a preferred embodiment, the present method comprises administering, within 24 hours of the occurrence of the trauma, a liver GTP increasing component selected from the group consisting of:
2-400 mg, more preferably 5-40 mg guanosine equivalents;
3-10 g ribose equivalents, more preferably 2-10 g D-ribose equivalents;
and combinations thereof.

Liver GTP may be increased further by employing folic acid or equivalents thereof. Thus, in a preferred embodiment the present method comprises enterally administering, within 24 hours of the trauma, 0.1-10 mg, preferably 0.2-5 mg folic acid equivalents. The term “folic acid equivalents” encompasses folic acid as well as salts of folic acid and precursors of folic acid or folic acid salts, notably precursors that can liberate folic acid, a folic acid salt or a metabolically active form of folic acid by in vivo conversion, e.g. hydrolysis, of the precursor molecule. Examples of suitable precursors include tetrahydrofolic (tetrahydropteroyl) polyglutamate, tetrahydrofolic glutamate, and 5-methyl and/or 10-methyl substituted analogues thereof. The folic acid equivalents according to the present invention may also comprise a pteroyl group in the dihydro form, be it that it is preferred to use the tetrahydro form.

The inclusion of folic acid in the indicated concentration range provides support for the biosynthesis of GTP. Another advantage of the use of folic acid in accordance with the present invention is the favourable impact on ADMA plasma concentrations (see below)²⁹. A combination of folic acid and ribose is particularly effective in maintaining/restoring liver GTP levels. Hence, in a particularly preferred embodiment the present method employs a combination of folic acid and ribose.

In a particularly preferred embodiment, the present method comprises administering, within 24 hours of the occurrence of the trauma, 2-100 mg guanosine equivalents, more preferably 5-40 mg guanosine equivalents. Guanosine is a precursor of GTP. Unexpectedly, the inventors have discovered that other potential precursors of GTP, e.g. guanine and guanosine monophosphate (GMP) are far less suitable.

In a very preferred embodiment of the invention, the present method comprises administering, within 24 hours of the occurrence of the trauma, 0.1-50 mmoles of peptides with ACE inhibiting activity, said peptides exhibiting an IC-50 concentration of less than 1000 μM. Although the inventors do not wish to be bound by theory, it is believed that ACE inhibitors may be able to increase the bio-availability of NO to endothelial cells, thereby improving endothelial function. ADMA is cleared by excretion into urine and by metabolisation by dimethylarganine dimethylaminohydrolase, which is abundantly expressed in liver and kidney, and also in endothelial cells. It is hypothesised that the clearance function of both liver and kidney is mediated by the endothelial cells in these highly vascularised organs. Thus, the vitality of the endothelial cells would be vital for the clearance of ADMA. This hypothesis is further supported by the observation that ADMA levels are usually increased in subjects with vascular diseases or with risk factors for vascular diseases, such as hypercholesterolemia and hypertension. In all these conditions endothelial function is compromised, whereas liver and kidney function are often unaffected.

In yet another advantageous embodiment of the invention the present method comprises co-administering, within 24 hours of the occurrence of the trauma, flavonoids in an amount of 0.5-200 mg, preferably of 1-100 mg and most preferably of 5-50 mg. Flavonoids, such as luteolin, quercetin and apigenin, were found to be good xanthine oxidase inhibitors and to inhibit oxidative stress, as demonstrated by their effect on plasma concentrations of malon dialdehyde. In addition, flavonoids were also found to assist in the maintenance and restoration of liver GTP level. In a particularly preferred embodiment, the present composition contains flavonoids selected from the group consisting of luteolin, quercetin, apigenin and combinations thereof, preferably in a concentration of at least 2 mg/l, more preferably of at least 5 mg/l, most preferably of at least 10 mg/l.

Another aspect of the invention relates to aqueous liquid compositions for use in the present in the present method. More particularly, this aspect concerns an aqueous liquid composition suitable for enteral administration containing:
20-200 g/l digestible dissolved carbohydrates;
5-5000 mg/l guanosine equivalents and at least one of:
   1-100g/l ribose equivalents;
   2-2000 mg/l flavonoids; and
45 to 97.95 wt. % water.

In one preferred embodiment, the aqueous liquid composition contains at least 1-100 g/l ribose equivalents. In another preferred embodiment, the liquid composition contains 2-2000 mg/l flavonoids. Particularly preferred is a liquid composition containing guanosine equivalents, ribose equivalents and flavonoids in the indicated amounts.

Yet another aspect of the invention relates to an aqueous liquid composition suitable for enteral administration containing:
20200 g/l digestible dissolved carbohydrates;
0.01 to 10 mM of peptides with ACE inhibiting activity, said peptides exhibiting an IC-50 concentration of less than 1000 μM.; and
45 to 97.95 wt. % water.

In a particularly preferred embodiment, the aforementioned liquid composition additionally contains at least 5 mg/l guanosine equivalents. In another particularly preferred embodiment the liquid composition additionally contains at least 1 g/l, more preferably at least 3 g/l ribose equivalents. Typically the amount of ribose equivalents contained in the liquid composition will not exceed 100 g/l, preferably it does not exceed 50 g/l. In yet another advantageous embodiment of the invention the aqueous liquid composition contains flavonoids in a concentration of 2-2000 mg/l.

Preferably, the present composition contains a peptide with ACE inhibiting activity or a blend of such peptides in a concentration that is not below 10%, preferably not below 50% of the IC-50 concentration of said peptide or said blend of peptides. ACE inhibiting peptides may suitably be incorporated in the present composition in the form of protein hydrolysates, particularly milk protein hydrolysates.

The present liquid composition advantageously contains at least 10 mg/l guanosine equivalents. Generally, the concentration of guanosine equivalents in the composition will not exceed 2000 mg/l, preferably it will not exceed 1000 mg/l, more preferably it will not exceed 500 mg/l.

In another preferred embodiment, the liquid composition of the invention contains between 0.2 and 400 mg/l folic acid equivalents. More preferably, said composition contains between 0.5 and 100 mg/l folic acid equivalents.

Preferably, flavonoids are contained in the present composition in a concentration of at least 5 mg/l, more preferably of at least 10 mg/l. Usually, the flavonoid concentration will not exceed 1000 mg/l, preferably it does not exceed 500 mg/l. Flavonoids, such as luteolin, quercetin and apigenin, were found to be good xanthine oxidase inhibitors and to inhibit oxidative stress, as demonstrated by their effect on plasma concentrations of malon dialdehyde. In addition, flavonoids were also found to assist in the maintenance and restoration of liver GTP level. In a particularly preferred embodiment, the present composition contains flavonoids selected from the group consisting of luteolin, quercetin, apigenin and combinations thereof in a concentration of at least 2 mg/l, preferably of at least 5 mg/l.

For patients who find it difficult to swallow or who experience nausea etc., it is important that the digestible carbohydrates can be delivered in concentrated liquid form. Consequently, it is preferred to include the digestible water soluble carbohydrates in a concentration of at least 50 g/l, more preferably of at least 70 g/l and most preferably at least 80 g/l.

In order to minimise the risk of regurgitation and also to minimise the residence time in the stomach, it is preferred that the liquid composition contains less than 30 g/l lipids, more preferably less than 20 g/l lipids and most preferably less than 10 g/l lipids. For similar reasons, also the protein level of the present composition is preferably relatively low, especially below 40 g/l. Another way to reduce the risk of regurgitation is to reduce the volume size of the serving (e.g. to less than 100 ml), or to apply a tube in the duodenum.

The present liquid composition may, for instance, take the form of a solution, a suspension or an emulsion. It is preferred to employ a liquid composition in the form of a solution that contains essentially no undissolved components, e.g. as demonstrated by the fact the liquid composition is clear and transparent.

Yet another aspect of the present invention relates to a composition that can be reconstituted with water to the present aqueous liquid composition. Typically, the reconstitutable composition can take the form of a liquid concentrate, a paste, a powder, granules, tablets etc. Preferably, the reconstitutable composition is a dry product, particularly a dry product with a moisture content of less than 10 wt. %, preferably of less than 7 wt. %.

### REFERENCES

1. Thomson A B, Keelan M, Thiesen A, et al. Small-bowel review: diseases of the small intestine. Dig Dis Sci 2001; 46(12):2555-66.
2. Baue A E. Nutrition and metabolism in sepsis and multisystem organ failure. Surg Clin North Am. 1991; 71(3):549-65.
3. Pugin J, Chevrolet J C. [The intestine-liver-lung axis in septic syndrome]. Schweiz Med Wochenschr 1991; 121(42):1538-44.
4. Matuschak G M. Liver-lung interactions in critical illness. New Horiz 1994; 2(4):488-504.
5. Tadros T, Traber D L, Herndon D N. Hepatic blood flow and oxygen consumption after burn and sepsis. J Trauma 2000; 49(1):101-8.
6. Towfigh S, Heisler T, Rigberg D A, et al. Intestinal ischemia and the gut-liver axis: an in vitro model. J Surg Res 2000; 88(2):160-4.
7. Zeuzem S. Gut-liver axis. Int J Colorectal Dis 2000; 15(2):59-82.
8. Yao Y, Yu Y, Wu Y, et al. The role of gut as a cytokine-generating organ in remote organ dysfunction after intestinal ischemia and reperfusion. Chin Med J (Engl) 1998; 111(6):514-8.
9. Nieuwenhuijzen G A, Deitch B A, Goris R J. The relationship between gut-derived bacteria and the development of the multiple organ dysfunction syndrome. J Anat 1996; 189(Pt 3):537-48.
10. Nieuwenhuijzen G A, Deitch R A, Goris R J. Infection, the gut and the development of the multiple organ dysfunction syndrome. Eur J Surg 1996; 162(4):259-73.
11. Baue A E. The role of the gut in the development of multiple organ dysfunction in cardiothoracic patients. Ann Thorac Surg 1993; 55(4):822-9.
12. Kaminski K A, Bonda T A, Korecki J, Musial W J. Oxidative stress and neutrophil activation—the two keystones of ischemia/reperfusion injury. Int J Cardiol 2002; 86(1):41-59.
13. Akcakaya A, Alimoglu O, Sahin M, Abbasoglu S D. Ischemia-reperfusion injury following superior mesenteric artery occlusion and strangulation obstruction. J Surg Res 2002; 108(1):39-43.
14. Canas P E. The role of xanthine oxidase and the effects of antioxidants in ischemia reperfusion cell injury. Acta Physiol Pharmacol Ther Latinoam 1999; 49(1):13-20.
15. Moore R M, Muir W W, Granger D N. Mechanisms of gastrointestinal ischemia-reperfusion injury and potential therapeutic interventions: a review and its implications in the horse. J Vet Intern Med 1995; 9(3):115-32.
16. Lai H S, Chen W J, Chiang L Y. Free radical scavenging activity of fullerenol on the ischemia-reperfusion intestine in dogs. World J Surg 2000; 24(4):4504.
17. Nakamura M, Ozaki M, Fuchinoue S, et al. Ascorbic acid prevents ischemia-reperfusion injury in the rat small intestine. Transpl Int 1997; 10(2):89-95.
18. Gunel E, Caglayan F, Caglayan O, et al. Treatment of intestinal reperfusion injury using antioxidative agents. J Pediatr Surg 1998; 33(10):1536-9.
19. Grynberg A, Demaison L. Fatty acid oxidation in the heart J Cardiovasc Pharmacol 1996; 28(Suppl 1):S11-7.
20. Longarela A, Olarra J, Suarez L, Garcia de Lorenzo A. [Metabolic response to stress, can we control it?]. Nutr Hosp 2000; 15(6):275-9.
21. Ma S W, Foster D O. Starvation-induced changes in metabolic rate, blood flow, and regional energy expenditure in rats. Can J Physiol Pharmacol 1986; 64(9):1252-8.
22. Bark T, Katouli M, Svenberg T, Ljungqvist O. Food deprivation increases bacterial translocation after non-lethal haemorrhage in rats. Eur J Surg 1995; 161(2):67-71.
23. Salman F T, Buyruk M N, Gurler N, Celik A. The effect of surgical trauma on the bacterial translocation from the gut. J Pediatr Surg 1992; 27(7):8024.
24. Nettelbladt C G, Katouli M, Volpe A, et al. Starvation increases the number of coliform bacteria in the caecum and induces bacterial adherence to caecal epithelium in rats. Eur J Surg 1997; 163(2):135-42.
25. Loegering D J, Feintuch J J. Depression of the reticuloendothelial system following graded isoproterenol-induced myocardial injury. Circ Shock 1979; 6(4):385-9.
26. Haglind E, Wang D, Klein A S. Hepatic reticuloendothelial system dysfunction after intestinal ischemia-reperfusion. Shock 1996; 5(1):72-5.
27. Klein A S, Zhadkevich M, Wang D, et al. Discriminant quantitation of postransplant hepatic reticuloendothelial function: The impact of ischemic preservation. Transplantation 1996; 61(8):1156-61.
28. Brengman M L, Wang D, Willins K B, et al. Hepatic killing but not clearance of systemically circulating bacteria is dependent upon peripheral leukocytes via Mac-1 (CD11/CD18). Shock 2003; 19(3):263-7
29 Holven K B, Haugstad T A, Holm T et al. Folic acid treatment reduces elevated plasma levels of asymetric dimethylarganine in hyperhomocysteinaemic subjects. Br J Nutr 2003 89(3); 359-63
   Methods
   Determination of the IC-50 Concentration

The IC-50 concentration as referred to in this application is the concentration at which a substance reduces the activity of angiotensin converting enzyme (ACE) by 50%, using the testing conditions as described below.

ACE is capable of cleaving a substrate, FAPGG (N-[3-(2-furyl) acryloyl]-L-phenylalanylglycylglycine), into FAP and GG. The intact substrate is spectrophotometrically detectable at a wavelength of 340 nm. The cleavage products are not detectable at said wavelength. The absorbance of an aqueous solution of the substrate to which ACE is added will decrease over time as result of the enzymatic cleavage of the substrate. In order to assess the ACE inhibiting properties of substances, these substances are introduced into the ACE-substrate mixture at different concentrations. The absorbance at 340 nm is followed for 20 minutes and the rate at which the absorption decreases is calculated from this data.

The method employs positive and negative controls for calibration.
Negative control: ACE and FAPPG (without test substance)
Positive control: ACE/FAPGG and pharmacological ACE inhibitor (e.g. Captotril, 25 nM)
   Materials:

96 wells plate

Microplate reader (340 nm filter; kinetic protocol)

Angtiotensin Converting Enzyme, 0.16 mU/μl, ex Sigma® (A-6778)

FAPGG, 2 mg/ml (5 mM) ex Sigma® (F-7131)

ACE-buffer: 17.6 mg/ml (300 mM) NaCl, 12 mg/ml (50 nM Hepes, pH 7.5

Method:

Adjust the incubator of the microplate reader to 37° C.

Dissolve and dilute the test components in the ACE buffer

Pipet 60 μl per well of the samples (including positive and negative controls) in the 96-wells plate

Add 30 μl per well of FAPGG (2 mg/ml in ACE buffer)

Incubate the plate at 37° C. for 5 minutes

Ad 10 μl per well of ACE (0.16 mU/μl)

Measure the absorbance at 340 nm for 20 minutes (kinetic protocol, 80 readings, one reading every 15 seconds)

### EXAMPLES

### Example 1

An aqueous liquid composition to be administered in a serving of 200 ml, comprising per 100 ml:

| | | |
|---|---|---|
| Glucose | 1 | g |
| Maltodextrin DE 5 | 10 | g |
| Guanosine | 5 | mg |

The liquid is to be administered in two servings within 24 hours of the occurrence of a trauma.

### Example 2

An aqueous liquid composition to be administered in a serving of 200 ml, comprising per 100 ml:

| | | |
|---|---|---|
| Glucose syrup DE 12 | 11.5 | g |
| Glucose | 2 | g |
| Folic acid | 100 | μg |
| Guanosine | 2 | mg |

The liquid is to be administered in three servings within 24 hours of the occurrence of a trauma.

### Example 3

An aqueous liquid composition to be administered in a serving of 200 ml, comprising per 100 ml:

| | | |
|---|---|---|
| Dextrin | 11.5 | g |
| Glucose | 2 | g |
| Folic acid | 100 | μg |
| αₛ₁-Casein hydrolysate ^{#} | 7 | g |

^{#} Ex DMV International; containing 6% C12 peptide

The liquid is to be administered in four servings within 24 hours of the occurrence of a trauma.

### Example 4

An aqueous liquid composition to be administered in a serving of 125 ml, comprising per 100 ml:

| | | |
|---|---|---|
| Glucose syrup DE 19 | 11.5 | g |
| Glucose | 2 | g |
| Folic acid | 200 | μg |
| Casein hydrolysate ^{$} | 1.75 | g |

^{$} containing 0.05 g (76 μmole) ACE inhibiting peptide with IC-50 of 6 μM

The liquid is to be administered in four servings within 24 hours of the occurrence of a trauma.

### Example5

An aqueous liquid composition to be administered in a serving of 200 ml, comprising per 100 ml:

| | | |
|---|---|---|
| Maltose | 1 | g |
| Glucose syrup DE 29 | 10 | g |
| Folic acid | 200 | μg |
| GTP | 5 | mg |
| Ribose | 1 | g |
| Soy protein hydrolysate ^{@} | 2 | g |

^{@} containing at least 0.1 g ACE inhibiting peptide with IC-50 of less than 200 μM

The liquid is to be administered in two servings within 24 hours of the occurrence of a trauma.

### Example 6

An aqueous liquid composition to be administered in a serving of 500 ml, comprising per 100 ml:

| | | |
|---|---|---|
| Maltose | 1 | g |
| Glucose syrup DE 32 | 10 | g |
| Folic acid | 50 | μg |
| GTP | 1 | mg |
| Ribose | 0.5 | g |
| αₛ₁-Casein protein hydrolysate * | 2 | g |

* ex DMV International; containing 8% C12 peptide

The liquid is to be administered in two servings within 24 hours of the occurrence of a trauma by means of tube feeding.

### Example 7

A powder formulation to be reconstituted to a single serving with 200 ml water:

| | | |
|---|---|---|
| Dextrin | 23 | g |
| Glucose | 4 | g |
| Folic acid | 200 | μg |
| Casein hydrolysate ^{#} | 1.74 | g |

^{#} containing 0.05 g ACE inhibiting peptide with IC-50 of 5 μM

The reconstituted liquid is to be administered four times within 24 hours of the occurrence of a trauma.

### Example 8

Rat studies were carried out to elucidate whether pre-operative supplementation with carbohydrates improves post-operative organ function and decreases multiple organ dysfunction-associated risk factors.

Methods:

One group of male wistar rats were fasted for 16 hours (water ad libitum), prior to clamping the SMA. The intervention group received 113 g of dextrin and 12.7 g fructose per litre, plus an isotonic mix of salts and citric acid in drinking water, starting 5 days before the operation and continued until the day of operation. Ad libitum water served as control. The animals were sacrificed by exsanguination; intestinal permeability and translocation of bacteria were measured immediately, plasma and different organ samples were frozen in liquid nitrogen, for organ function parameters measurements. Sham-fasted animals served as controls.

Results—Intestine

Ischemia reperfusion in the fasted animals resulted in a significant increased intestinal permeability (FIG. 1). Preoperative ad libitum administration of a carbohydrate drink showed to preserve a significantly (P<0.05) better intestinal barrier function when compared to overnight fasted ischemic rats (FIG. 1).

[image]

Results—Bacterial Translocation

Fasted operated rats showed an increased bacterial translocation to the liver, kidney and mesenteric lymph nodes (FIG. 2A-C.) when compared to sham fasted rats or sham fed rats. Preoperative supplementation of the carbohydrate drink significantly decreased bacterial translocation to the liver, kidney and mesenteric lymph nodes (FIG. 3A-C.) as compared to IR fasted animals. Furthermore, a trend (P=0.07) to decreased bacterial translocation was seen in the spleen of preoperative fed animals (FIG. 2D.).

[image]

[image]

Results—Lung

The lung, showed increased neutrophil infiltration as indicated by m myeloperoxidase activity (FIG. 3A.) in the IR fasted group when compared with the sham-fasted group. The group, pre-operatively supplemented with the carbohydrate mixture showed a significant (P<0.02) decrease when compared to the IR fasted rats (FIG. 2A.). Moreover, the IR fasted group showed significantly (P=0.014) decreased GSH concentration compared to the pre-operative supplemented group. In contrast, the GSH concentration of the IR supplemented group was almost retained at the level of the sham fasted animals (FIG. 3B). Oxidative stress, indicated as MDA concentration, showed a trend (P<0.1) to decrease when compared to IR fasted animals.

[image]

Results—Systemic Parameter

Rats that were allowed ad libitum pre-operative access to the carbohydrate drink showed a significant (P=0.028) decrease in urea concentration when compared to IR fasted rats (FIG. 4).

[image]

Results—Plasma ADMA and IL-6 Concentration

Asymmetrical dimethylarginine (ADMA) concentration, recently suggested to be a risk factor for organ dysfunction, was significantly increased in the IR fasted rats (P<0.02) as compared to sham-fasted (FIG. 5A.). Importantly, the pre-operative supplemented group showed significantly (P<0.01) decreased ADMA concentration and were shown to be deprived from an increase in ADMA when compared to IR fasted and sham-fasted animals respectively (FIG. 5A.) Another parameter that has concentration-dependently been linked to the incidence and severity of single and multiple organ dysfunction ((S)MOD) is IL-6, a pro-inflammatory cytokine. The IL-6 concentration showed a significant P=0.02) decrease in the group pre-operatively supplemented with the carbohydrate mixture as compared to the IR fasted rats (FIG. 5B).

[image]

Conclusions

In conclusion, pre-operative administration of carbohydrates decreased MOD. This decrease was shown by improved intestinal barrier function and lowered bacterial translocation. Furthermore, lung inflammation pulmonary oxidative stress and plasma urea were decreased. These improvements in organ function parameters in the carbohydrate-fed rats were paralleled by a simultaneous decrease in ADMA and IL-6 concentration. The beneficial effects of preoperative carbohydrate supplementation on decreasing MOD and MOD associated factors suggest an important role for preoperative nutrition to improve post-operative recovery.

### Example 9

Rat studies were conducted using the model described in example 8. Intervention groups were allowed either ad libitum presurgical feeding or ad libitum presurgical feeding with an additional flavonoid mixture added to the feeding. To 15 kgs of the latter feed 4 g of quercetine, 3 g of luteoline, 3 g apigenince, 5 g epicatechine and 10 green tea extract had been added. Liver GTP levels, plasma creatinine and plasma urea levels were determined after exsanguinations. The results obtained are depicted in FIGS. 6-8.

[image]

As can be deduced from FIG. 6, liver GTP increased in fed IR fasted animals compared to IR fasted animals and further increased in IR fed+flavonoid rats (p<0.05).

[image]

FIG. 7 shows that kidney function is improved in ischemia reperfusion fed animals compared to ischemia-reperfusion fasted animals and further improved in ischemia reperfusion fed animals additionally fed with flavonoids, back to sham levels (p<0.05).

[image]

FIG. 8 shows that plasma urea levels improved in ischemia reperfusion fed animals compared to ischemia-reperfusion fasted animals and further improved in Ischemia-reperfusion fed animals additionally fed with flavonoids (p<0.05).

### Example 10

HepG2 cells, a human hepatocarcinoma cell line, were obtained from ATCC. These were maintained in MEM supplemented with 10% FCS; 1% NEAA; 1% penicillin/streptomycin mixture. Cells were seeded primarily at a density of approximately 1-2×10⁶ cells and were split and transferred to new flasks when showing 70-90% confluency. 96-well microtitre plates (ex Micronic, Leylstad, N L.), containing 0.35×10⁶ cells per well were incubated for 24 hours at 37° C.; 5% CO₂. HepG2's were folic acid challenged by addition of folic acid free media for 1 hour. This folic acid challenged cells were compared with cells that remained at an increasing concentration of folic acid or ribose.

Nucleotide Measurements

Nucleotides were measured as described by van Hoorn et al., Analytical Biochemistry (2003), 320, 82-87.

Experiment Conducted for this Study:

1. 6.5 hours incubation of HepG2 cells in either folic acid depleted media or media containing 2.27 μM folic acid

This experiment showed that HepG2 cells in presence of 2.27 μM folic acid significantly increased the GTP concentration when compared to folic acid challenged cell as can be seen in FIG. 9.

[image]

In a similar experiment it was shown that ribose had similar GTP increasing effects and moreover that ribose could have an additive effect on the effect of folic acid (FIG. 10)

[image]

1-18. (canceled)

19. A method of preventing multiple organ dysfunction in a mammal suffering from trauma, comprising enterally administering to said mammal an aqueous liquid composition comprising digestible water soluble carbohydrates and a liver guanosine-5′-triphosphate (GTP) increasing component within 24 hours of the occurrence of the trauma, (i) the liver GTP increasing component selected from the group consisting of: 2-2000 mg guanosine equivalents; 0.5-40 g ribose equivalents; and combinations thereof; and (ii) at least 20 g of the digestible water soluble carbohydrates in the form of the aqueous liquid composition containing at least 10 g/l of said digestible water soluble carbohydrates.

20. The method according to claim 19, further comprising administering, within 24 hours of the occurrence of the trauma, 0.05-100 mmole of peptides with Angiotensin Converting Enzyme (ACE) inhibiting activity, said peptides exhibiting an IC-50 concentration of less than 1000 μM.

21. A method of preventing multiple organ dysfunction in a mammal suffering from trauma, comprising enterally administering an aqueous liquid composition comprising digestible water soluble carbohydrates; and (i) 0.05-100 mmole of peptides with ACE inhibiting activity within 24 hours of the occurrence of the trauma, said peptides exhibiting an IC-50 concentration of less than 1000 μM; and (ii) at least 20 g of the digestible water soluble carbohydrates in the form of the aqueous liquid composition containing at least 10 g/l of said digestible water soluble carbohydrates.

22. The method according to claim 21, further comprising administering, within 24 hours of the occurrence of the trauma, a liver GTP increasing component selected from the group consisting of: 2-2000 mg guanosine equivalents; 0.1-10 g folic acid equivalents; 0.5-40 g ribose equivalents; and combinations thereof.

23. The method according to claim 19, wherein the trauma is surgery.

24. The method according to claim 23, wherein the surgery is prescheduled surgery.

25. The method according to claim 19, wherein the liquid composition is administered prior to the occurrence of the trauma.

26. The method according to claim 19, wherein the liquid composition contains between 30 and 200 g/l of digestible polysaccharides.

27. The method according to claim 19, wherein the digestible water soluble carbohydrates are selected from the group consisting of dextrins, maltodextrins, starches, dextran and combinations thereof.

28. The method according to claim 19, wherein at least 50 g of the digestible water soluble carbohydrates is enterally administered in the form of the aqueous liquid composition.

29. The method according to claim 19, wherein 2-2000 mg guanosine equivalents are enterally administered within 24 hours of the occurrence of the trauma.

30. An aqueous liquid composition suitable for enteral administration, comprising:
20-200 g/l digestible dissolved carbohydrates;
5-5000 mg/l guanosine equivalents;
at least one of 1-100 g/l ribose equivalents and 2-2000 mg/l flavonoides; and
45 to 97.95 wt. % water.

31. The aqueous liquid composition according to claim 30, wherein the aqueous liquid composition is comprised of 5-5000 mg/l guanosine equivalents and at least 1-100 g/l ribose equivalents.

32. An aqueous liquid composition suitable for enteral administration, comprising:
20-200 g/l digestible dissolved carbohydrates;
0.01 to 10 mM of peptides with ACE inhibiting activity, said peptides exhibiting an IC-50 concentration of less than 1000 μM;
at least one of:
5-5000 mg/l guanosine equivalents;
1-100 g/l ribose equivalents;
0.2 and 400 mg/l folic acid equivalents;
2-2000 mg/l flavonoides; and
45 to 97.95 wt. % water.

33. The aqueous liquid composition according to claim 32, wherein the composition contains 5-5000 mg/l guanosine equivalents and/or 1-100 g/l ribose equivalents.

34. The aqueous liquid composition according to claim 30, further comprising between 0.2 and 400 mg/l folic acid equivalents.

35. The aqueous liquid composition according to claim 30 suitable for enteral administration, further comprising 0.01 to 10 mM of peptides with ACE inhibiting activity, said peptides exhibiting an IC-50 concentration of less than 1000 μM, wherein the liquid composition is a clear aqueous solution.

36. A composition that can be reconstituted with water to a liquid composition according to claim 30.
